(19) United States
(12) Patent Application Publication
(10) Pub. No.: US 2014/0066731 A1
(43) Pub. Date: Mar. 6, 2014

(54) Batteryless Portable Medical Devices

(71) Applicant: HCL Technologies Limited, Chennai (IN)

(72) Inventor: Siva Sakthivel Sadasivam, Bangalore (IN)

(73) Assignee: HCL Technologies Limited, Chennai (IN)

(21) Appl. No.: 14/018,936

(22) Filed: Sep. 5, 2013

(30) Foreign Application Priority Data
Sep. 5, 2012 (IN) 3681/CHE/2012

(51) Int. Cl. A61B 5/00

(52) U.S. Cl. CPC A61B 5/6887
USPC 600/323; 600/300; 600/365

(57) ABSTRACT

This embodiment relates to portable handheld medical devices and in particular to handheld devices functioning without batteries. The medical devices connect to an external system and receive power. The external system may refer to any of everyday devices like a laptop or a mobile phone and so on. The medical devices operate once the power source i.e. the external device is connected. The medical devices can be shut down after use and connection between the external system and handheld medical devices is removed.

### PRIORITY DETAILS

The present application is based on, and claims priority from, Indian Application Number 3681/CHE/2012, filed on 5th Sep. 2012, the disclosure of which is hereby incorporated by reference.

### TECHNICAL FIELD

This embodiment relates to portable handheld medical devices and in particular to handheld devices functioning without batteries.

### BACKGROUND

Handheld portable medical devices have revolutionized the patient monitoring and health care industry in a big way. The hospital as well as patients can easily monitor their health using suitable handheld medical devices. And with the help of advances in communication technology one can keep log of readings taken by a device, communicate changes to medical service provider and also prompt for immediate action in case of an emergency.

Most of the handheld portable medical devices have a battery acting as their power source. Some devices make use of lightweight, slow discharging batteries for longer life. Some use rechargeable batteries, which need to be connected to an external power source for charging. The use of batteries increases the volume and weight of the handheld portable medical device. In addition, most batteries end up in landfills polluting the environment.

### OBJECT OF EMBODIMENT

The principal object of the embodiment is to provide a batteryless portable medical device.

Another object of the embodiment is to use power supply from a commonly used external device similar to a USB port.

### STATEMENT OF EMBODIMENT

Accordingly the embodiment provides a batteryless handheld portable medical system for monitoring physiological parameters. The system contains a handheld portable medical device and an external device for powering the handheld portable medical system. The external device powers the handheld portable medical device using a medical grade USB cable or any similar cable. The external device may be a commonly used device like a laptop or a mobile phone.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

This embodiment is illustrated in the accompanying drawings, throughout which like reference letters indicate corresponding parts in the various figures. The embodiments herein will be better understood from the following description with reference to the drawings, in which:

FIG. 1 illustrates a broad block diagram showing the connection between an external device and a portable medical device, according to the embodiments disclosed herein;

FIG. 2 illustrates a block diagram showing the connectivity between an external device and a portable medical device through an power cable, according to the embodiments disclosed herein; and

FIG. 3 is a flowchart describing the process of using a handheld portable medical device without batteries, according to the embodiments as disclosed herein.

### DETAILED DESCRIPTION OF EMBODIMENT

The embodiments herein, the various features, and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

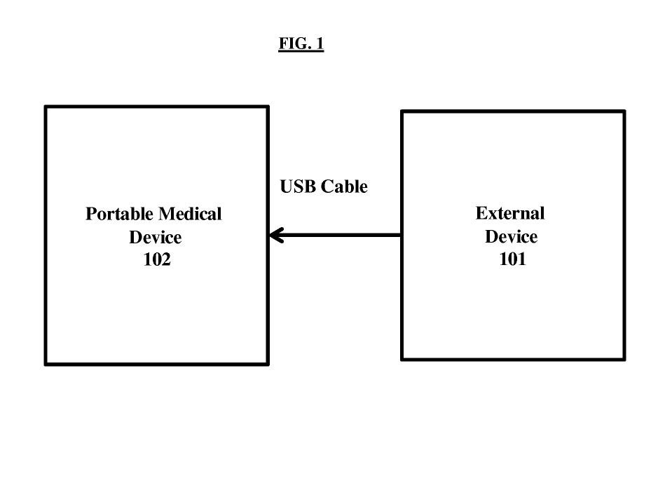

FIG. 1 illustrates a broad block diagram showing the connection between an external device and a portable medical device, according to the embodiments disclosed herein. An external device 101 is connected to the portable medical device 102 using a USB cable. The portable medical device 102 may be any handheld device like blood sugar monitor or a blood pressure or pulse oxymeter, similar device. The external device 101 may be any commonly used device from which power can be drawn. To draw power, a USB port in an external device like a mobile phone or a laptop may be used. A medical grade USB cable may also be used. The portable medical device 102 does not contain batteries and draws power from the external device 101 when connected. Once the portable medical device 102 is connected to the external device 101 and power of the external device is ON, the portable medical device 102 may be switched ON and used. After using the portable medical device 102, the external device 101 can be disconnected; thereby disconnecting power supply to the portable medical device 102. Not using batteries in the portable medical device 102 makes it smaller in size, reduces cost to OEM.

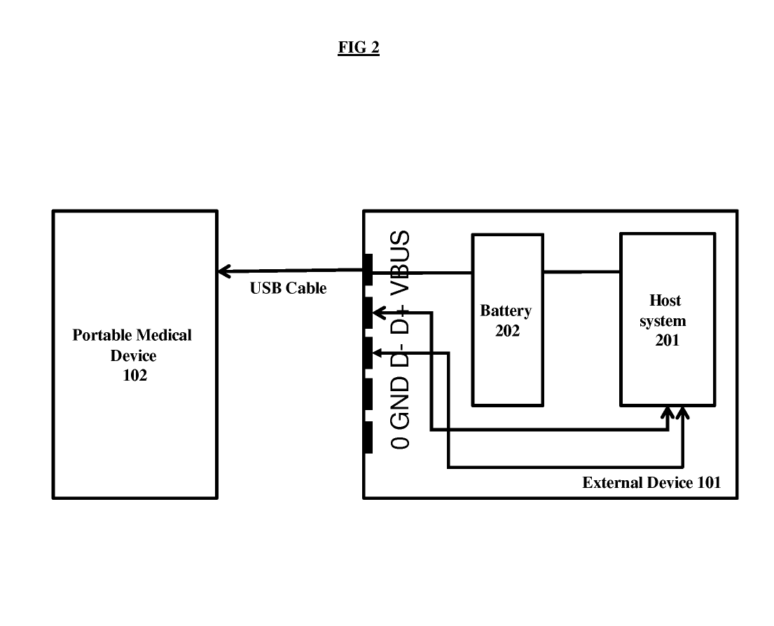

FIG. 2 illustrates a block diagram showing the connectivity between an external device and portable medical device through a USB cable, according to the embodiments disclosed herein. The USB port with all the PINS is shown in the external device 101. The external device comprises of a host system 201 and a battery 202. The battery 202 powers both the host system and VBUS pin of the USB port. The portable medical device 102 connects to the host system 101/external device via a USB cable. The VBUS port of the USB cable supplies power to the portable medical device. As most of the portable medical device require vey little power for operation, the current supplied by the VBUS pin of the USB port is sufficient.

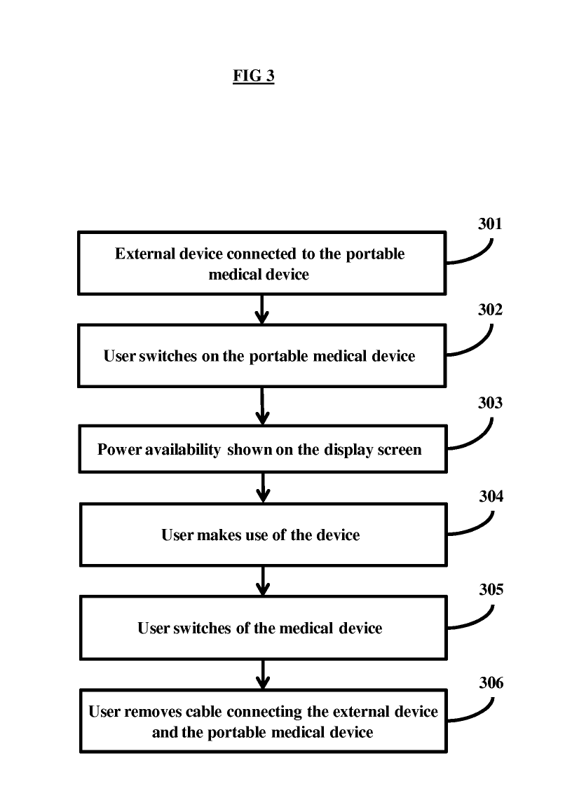

FIG. 3 is a flowchart describing the process of using a handheld portable medical device without batteries, according to the embodiments as disclosed herein. The external device 101 is connected (301) to the portable medical device 102 using a USB cable. This USB cable is a medical grade USB cable. User then switches ON (302) the portable medical device 102. The display screen of the portable medical device 102 shows 303-power availability using an icon. The user then makes use (304) of the portable medical device 102. After using, the user switches OFF (305) the portable medical device 102 and removes (306) the USB cable connecting the external device and the portable medical device.

The above embodiments disclose use of a USB cable, however, any other cable capable of powering a device by drawing power from another device may be used.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A handheld portable medical system for monitoring physiological parameters, said system comprising of:
a handheld portable medical device; and
an external device powering said handheld portable medical device,
wherein said handheld portable medical device does not include a power source.

2. The batteryless handheld portable medical system of claim 1, wherein said handheld portable medical device may be a blood glucose meter.

3. The batteryless handheld portable medical system of claim 1, wherein said handheld portable medical device may be a Pulse oxymeter.

4. The batteryless handheld portable medical system of claim 1, wherein said external device may connect to the handheld portable medical device using a medical grade USB cable.
